(19) United States
(12) Patent Application Publication
(10) Pub. No.: US 2005/0142624 A1
(43) Pub. Date: Jun. 30, 2005

(54) Chip and method for analyzing enzyme immunity

(76) Inventors: Takehiko Kitamori, Tokyo (JP); Manabu Tokeshi, Kanagawa (JP); Kiichi Sato, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/502,256

(22) PCT Filed: Nov. 8, 2002
(86) PCT No.: PCT/JP02/11679

(30) Foreign Application Priority Data
Jan. 24, 2002 (JP) 2002-16203

(51) Int. Cl. G01N 33/53; G01N 33/537; G01N 33/543; C12M 1/34

(52) U.S. Cl. 435/7.92; 435/287.2

(57) ABSTRACT

An enzyme immunoassay chip having, as micro channels, a reaction liquid leading-in flow passage part, a reaction flow passage part, and detection flow passage part sequentially disposed on a substrate continuously to each other, comprising an installed part for bead-bodies supporting antibodies and the bead-body flow stopping part formed in the micro channel of the reaction flow passage part, wherein enzyme reactive product flowing beyond the flow stopping part can be analyzed by using the chip.

### TECHNICAL FIELD

The present invention relates to an enzyme immunoassay chip and method. More specifically, the present invention relates to a novel microchip capable of executing the enzyme analysis efficiently with the high accuracy on the microchip, and an analysis method using the same.

### BACKGROUND ART

Conventionally, the immunoassay has been known as one of the important analyzing methods in the fields of medicine, biochemistry, or the like. However, according to the conventional methods such as the enzyme-linked immunosorbent assay (ELISA), the time of one day or more is needed to analyze, and moreover, a problem is involved in that the operation is complicated and the reagent cost is high. Accordingly, the present inventors have integrated the immunoassay method onto a microchip as one of the methods based on the achievement and the knowledge of integrating various chemical systems by the use of a microchip with a micro channel (fine groove) of the μm order on a substrate such as a glass chip, utilizing the short diffusion moving distance and the large specific interface area as the characteristics thereof so far. As a result thereof, an analysis method of measuring the polystyrene bead surface with a thermal lens microscope: TLM with a gold colloid as the label has already been developed. Thereby, shortening of the analysis time and reduction of the reagent have been realized. However, according to this method, since the minute surface of a sphere is measured, a problem is involved in that irregularity varies largely per each measurement point, the dynamic range is narrow and the skill is required for the measurement.

An object of the present invention is to provide a novel enzyme immunoassay microchip capable of solving the above-mentioned problems of the conventional technique and executing the immunoassay efficiently with the high accuracy, and an analyzing method using the same.

### DISCLOSURE OF THE INVENTION

The present invention has been completed by integrating an enzyme immunoassay system for coloring and measuring a substrate solution with an enzyme used as the label in a microchip based on the concept of solving the above-mentioned problems by providing a system for measuring a liquid phase, which can be measured relatively easily instead of the bead surface as the countermeasure for solving the above-mentioned problems.

That is, the present invention firstly provides an enzyme immunoassay chip comprising a reaction liquid leading-in flow passage part, a reaction flow passage part and a detection flow passage part disposed successively as a micro channel communicating with each other on a substrate, characterized in that the reaction flow passage part micro channel is provided with an inlet part for bead-bodies supporting antibodies, and a flow stopping part for the bead-body.

It secondly provides the above-mentioned enzyme immunoassay chip, characterized in that the width or the depth of the reaction flow passage part is sufficiently narrow or shallow for stopping the flow of the bead-body at the flow stopping part of the bead-body with the antibody supported, and it thirdly provides the enzyme immunoassay chip, characterized in that a plurality of the reaction flow passage part micro channels disposed side by side communicate with a detection flow passage part micro channel on the front side with respect to the detection point.

Then, the present invention fourthly provides an enzyme immunoassay method using an analysis chip of the present invention according to the above-mentioned first to third aspects, characterized in that enzyme reaction products produced by the antigen antibody reaction with the enzyme in the reaction flow passage part micro channel as the label is tested by the detection flow passage part, it fifthly provides the enzyme immunoassay method, characterized in that the enzyme reaction product is detected without contact, and it sixthly provides the enzyme immunoassay method, characterized in that the enzyme reaction product is detected by a thermal lens microscope system.

### BRIEF DESCRIPTION OF DRAWINGS

FIG. 1 is a perspective view and the essential part vertical cross sectional view schematically showing an example of the configuration of an analysis chip of the present invention.

FIG. 2 is a plan view showing another example of the arrangement of the micro channel.

FIG. 3 is a calibration curve of the example 2.

### BEST MODE FOR CARRYING OUT THE INVENTION

The present invention has the above-mentioned characteristics, and the embodiment thereof will be explained hereinafter.

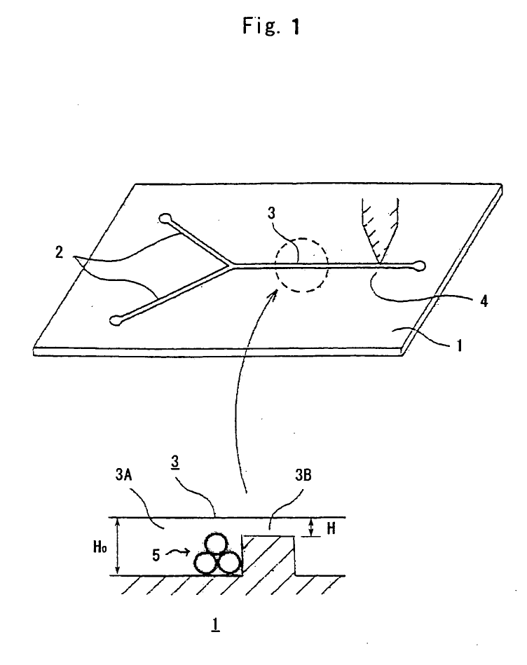

First, an enzyme immunoassay chip of the present invention will be explained according to the example schematically showing in FIG. 1. In a microchip having a reaction liquid leading-in flow passage part (2), a reaction flow passage part (3) and a detection flow passage part (4) arranged successively on a substrate (1) made of a glass, a silicon, a resin, or the like as the micro channel (fine groove), communicating with each other, the reaction flow passage part (3) is provided with an inlet part (3A) of a bead-body (5) for supporting an antibody, and a flow stopping part (3B) for stopping the flow (movement) of the bead-body (5) to the downstream area.

In the embodiment of FIG. 1, the flow stopping part (3B) has its depth (H) shallower than the depth (H₀) of the micro channel of the reaction flow passage part (3) so as to stop the flow of the bead-body (5).

As to the flow stopping part (3B), not only the method of adjusting the depth of the micro channel as in this example, but various countermeasures of providing a structure of stopping the flow of the bead-body (5) by narrowing the width (W) of the micro channel, or the like can be adopted. It is also possible to use a magnetic bead-body and providing the flow stopping part (3B) according to the arrangement of an external magnetic field applying means.

For example, in order to stop the bead-body by the depth (H) or the width (W) by the adjustment of the micro channel, the relationship with respect to the size (D) of the bead-body is determined in consideration of the introduction amount (volume) of the bead-body to be introduced into the micro channel inlet part (3A), the specific gravity thereof, the liquid flow rate in the micro channel, or the like. For example, as a common standard, H<D, W<D can be considered, however, H<(⅔)D, W<(⅔)D can be considered more preferably.

The reaction liquid leading-in flow passage part (2), the reaction flow passage part (3), and the detection flow passage part (4) can be formed by the conventional method such as etching by lithography, or the like. The same can be applied to the adjustment of the depth (H) and the width (W). The ordinary depth and width of the flow passage part micro channels can be determined according to the purpose, the kind of the subject, and the reaction. For example, a 500 μm or less width and a 300 μm or less depth can be presented as the common standards.

The conventionally known integration methods for a micro chip such as a method of providing an introduction groove hole part to the top end of the reaction liquid leading-in flow passage part (2), and a discharging groove hole part to the end of the detection flow passage part (4), or the like can be adopted optionally. The same is applied to the lamination of a cover plate on the substrate (1), or the like.

For example, by the use of the analysis chip of the present invention as mentioned above, the coloring, or the like by the reaction of the substrate solution flowing beyond the flow stopping part (3B) can be the measurement subject with the enzyme as the label without having the bead surface as the measurement subject as in the conventional configuration so that the enzyme-linked immunosorbent assay (ELISA) can be enabled easily and efficiently with the high accuracy.

Since introduction of bubbles to the micro channel and the bead-body inlet part at the time of the reaction and analysis is not preferable in terms of the analysis, for example a method of providing a minute hole or a minute exhausting channel in the transparent cover body or the chip substrate to be disposed on the chip surface in the upper part of the micro channel, a method of restraining the introduction of the bubbles at the time of supplying a specimen or a reagent to one of the micro channel paths having a Y shaped planar shape so as to allow inflow thereof to the other micro channel path, or the like, and the channel design therefor can be considered. A method of eliminating the bubbles by the vibration, the agitation, or the like of the bead-body can be considered.

Moreover, in the case introduction of a certain amount of beads is needed for the quantitative analysis, judgment by the volume, that is, the channel length according to the channel design is considered instead of counting the number of the beads.

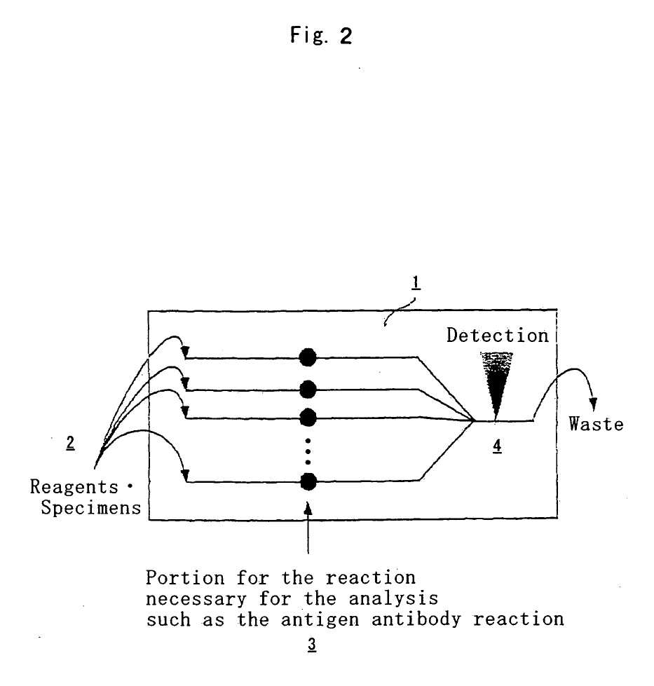

Of course FIG. 1 is for explaining the basic configuration of the microchip, and thus it is not limited thereto. For example, a plurality of reaction flow passage parts and a plurality of detection flow passage parts can be arranged on a substrate (1). Furthermore, as shown in FIG. 2, a detection point can be provided, and the detection flow passage part (4) with a plurality of reaction flow passage parts (3) disposed parallel each communicating therewith. In this case, for the purpose of the analysis of different kinds at the same time, after first introducing reagent solutions needed for the reaction simultaneously to the channels of each reaction flow passage parts (3) for the simultaneous reaction, the enzyme reaction substrate solution is introduced successively for each channel so as to detect the reaction product on the downstream side of the junction part.

Since the analysis results of the each channels can be measured at one detection point without the need of preparing a plurality of detector or moving the detector or the chip, analysis can be enabled easily and quickly.

For the detection for the immunoassay, it can be executed for example optically without contact. For example, the thermal lens microscope (TLM), which has been developed by the present inventors can be used effectively.

According to the present invention, the reaction product can be measured easily in the liquid phase by using an enzyme as the label substance and introducing a resin bead member supporting an antibody into the micro channel so as to stop the flow thereof.

Hereinafter, with reference to the examples, the present invention will be explained in further detail. Of course the invention is not limited by the following examples.

### EXAMPLES

### Example 1

A micro channel having a Y shaped plan arrangement provided with a stopping part (3B) having a 100 μm depth. (H₀) and a 250 μm width, with only the central part depth (H) made to 10 μm for stopping the beads was produced on a 3 cm×7 cm quartz glass substrate as shown in FIG. 1. Into the micro channel, about 50 μm diameter polystyrene beads with a human interferon gamma (IFN-γ) antibody fixed preliminarily as the reaction solid phase were introduced so as to execute the antigen antibody reaction, the washing operation, or the like in the chip. For the detection of the reaction product, a thermal lens microscope was used as the highly sensitive analysis method in the channel position as shown in FIG. 1.

Specifically, a specimen including IFN-γ of different concentrations, a biotinylated anti-IFN-γ, and a streptoavidin-peroxidase conjugate were provided by a pump successively for the reaction. After the reaction, a 4-AA (amino antipyrin) was supplied from one of the above-mentioned Y shaped micro channels and a TOOS and a H₂O₂ were supplied from the other one for the reaction with the enzyme. The product generated by the reaction, having the absorption local maximum wavelength at 550 nm was measured by the thermal lens microscope (excitation light beam: YAG laser 532 nm, probe optical conductor laser 670 nm) on the downstream side of the stopping part.

The IFN-γ was analyzed by the produced microchip enzyme immunoassay system so that a quantitative signal of the enzyme reaction product can be confirmed. Furthermore, in order to obtain a signal strength sufficient for the measurement, the optimum condition was sought with the concentration of the reagent, the flow rate and the reaction time changed. At the time of the antigen/antibody reaction, a good signal can be confirmed with a 1 μl/min flow rate and a 15 minute or more reaction time, and at the time of the measurement, with a 1×10⁻⁴M substrate concentration and a 0.1 μl/min or less flow rate. Under the conditions, the calibration curve of the signal strength with respect to the specimen concentration was produced. Compared with the analysis in the bulk, the analysis time was reduced from 2 days to 90 minutes, and the detection limit was about 8 digits so as to improve the detection limit by about 2 digits compared with the method of using a gold colloid label in the microchip.

Furthermore, the relationship of the temperature and the signal strength was examined so that the signal became maximum at about 50° C. so as to have the signal strength about 5 times as much as that of the room temperature. It was confirmed that the detection limit is further lowered by changing the temperature for raising the signal strength.

### Example 2

The quantitative analysis of the sex hormone 17β-estradiol as one kind of the endocrine disturbing substances, contained by a minute amount in an individual sea snail such as ibonishi was executed.

First, a micro channel having a 100 μm depth and a 250 μm width provided with a stopping part having a 10 μm depth for stopping the beads only in the central part was produced in a several cm square Pyrex glass substrate. With polystyrene beads having about 15 to 50 μm diameter introduced as the reaction solid phase into the chip, and the specimen and the various reagent solutions added thereto, the antigen antibody reaction, the washing operation, the enzyme reaction, or the like were executed in the chip. For the detection of the generated enzyme reaction product, the thermal lens microscope as a highly sensitive analysis method was used.

More specifically, after preliminarily introducing the beads with the 17β-estradiol antibody adsorbed into the micro channel of the produced chip, a solution as a mixture of a specimen including the 17β-estradiol and a 17β-estradiol labeled by a certain amount of a peroxidase was poured thereto by a syringe pump so as to execute the antigen antibody reaction competitively. After washing the unreacted product by a buffer, the quantitative analysis was executed by carrying out the enzyme reaction by introducing an enzyme substrate (4-amino antipyrin, N-hydroxy sulfopropyl aniline derivative, H₂O₂), and detecting the color developing substance produced thereby at the downstream part.

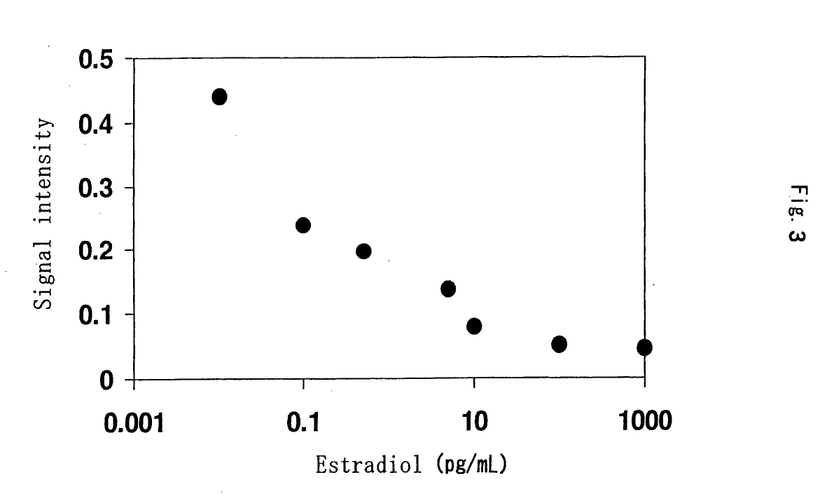

As a result, as shown in FIG. 3, the calibration curve can be produced in a relatively low concentration range up to 1,000 pg/mL. Also in consideration to the extremely small amount of the specimen volume needed for the assay, it was revealed that the sensitivity sufficient for the measurement by the extract liquid from an individual small snail can be provided.

### Industrial Applicability

As heretofore explained in detail, according to the present invention, the enzyme immunoassay can be carried out easily and efficiently with the high accuracy.

1. An enzyme immunoassay chip comprising a reaction liquid leading-in flow passage part, a reaction flow passage part and a detection flow passage part disposed successively as a micro channel communicating with each other on a substrate, characterized in that the reaction flow passage part micro channel is provided with an inlet part for bead-bodies supporting antibodies, and a flow stopping part for the bead-body.

2. The enzyme immunoassay chip according to claim 1, characterized in that the width or the depth of the reaction flow passage part is sufficiently narrow or shallow for stopping the flow of the bead-body at the flow stopping part of the bead-body with the antibody supported.

3. The enzyme immunoassay chip according to claim 1, characterized in that a plurality of the reaction flow passage part micro channels disposed side by side communicate with a detection flow passage part micro channel on the front side with respect to the detection point.

4. An enzyme immunoassay method using the analysis chip according to claim 1, characterized in that enzyme reaction products produced by the antigen antibody reaction with the enzyme in the reaction flow passage part micro channel as the label is tested by the detection flow passage part.

5. The enzyme immunoassay method according to claim 4, characterized in that the enzyme reaction product is detected without contact.

6. The enzyme immunoassay method according to claim 5, characterized in that the enzyme reaction product is detected by a thermal lens microscope system.

7. The enzyme immunoassay chip according to claim 2, characterized in that a plurality of the reaction flow passage part micro channels disposed side by side communicate with a detection flow passage part micro channel on the front side with respect to the detection point.

8. An enzyme immunoassay method using the analysis chip according to claim 2, characterized in that enzyme reaction products produced by the antigen antibody reaction with the enzyme in the reaction flow passage part micro channel as the label is tested by the detection flow passage part.

9. An enzyme immunoassay method using the analysis chip according to claim 3, characterized in that enzyme reaction products produced by the antigen antibody reaction with the enzyme in the reaction flow passage part micro channel as the label is tested by the detection flow passage part.

10. An enzyme immunoassay method using the analysis chip according to claim 7, characterized in that enzyme reaction products produced by the antigen antibody reaction with the enzyme in the reaction flow passage part micro channel as the label is tested by the detection flow passage part.

11. The enzyme immunoassay method according to claim 8, characterized in that the enzyme reaction product is detected without contact.

12. The enzyme immunoassay method according to claim 9, characterized in that the enzyme reaction product is detected without contact.

13. The enzyme immunoassay method according to claim 10, characterized in that the enzyme reaction product is detected without contact.

14. The enzyme immunoassay method according to claim 8, characterized in that the enzyme reaction product is detected by a thermal lens microscope system.

15. The enzyme immunoassay method according to claim 9, characterized in that the enzyme reaction product is detected by a thermal lens microscope system.

16. The enzyme immunoassay method according to claim 10, characterized in that the enzyme reaction product is detected by a thermal lens microscope system.

17. The enzyme immunoassay method according to claim 11, characterized in that the enzyme reaction product is detected by a thermal lens microscope system.

18. The enzyme immunoassay method according to claim 12, characterized in that the enzyme reaction product is detected by a thermal lens microscope system.

19. The enzyme immunoassay method according to claim 13, characterized in that the enzyme reaction product is detected by a thermal lens microscope system.
